(19) United States
(12) Patent Application Publication
(10) Pub. No.: US 2024/0383773 A1
(43) Pub. Date: Nov. 21, 2024

(54) MEASUREMENT DEVICE, WATER TREATMENT DEVICE, MEASUREMENT METHOD, AND WATER TREATMENT METHOD

(71) Applicant: Nomura Micro Science Co., Ltd., Atsugi-shi (JP)

(72) Inventor: Yukio NOGUCHI, Atsugi-shi (JP)

(73) Assignee: Nomura Micro Science Co., Ltd., Atsugi-shi (JP)

(21) Appl. No.: 18/544,640

(22) Filed: Dec. 19, 2023

(30) Foreign Application Priority Data
May 17, 2023 (JP) 2023-081881

(51) Int. Cl. C02F 1/00; C02F 1/70; C02F 1/72

(52) U.S. Cl. CPC C02F 1/008; C02F 1/70; C02F 1/72; C02F 2103/04; C02F 2209/22

(57) ABSTRACT

A measurement device irradiates water to be treated taken in from a main flow path with ultraviolet rays, and then measures an amount of dissolved oxygen after irradiation in the water to be treated, measures an amount of non-irradiated dissolved oxygen in the water to be treated taken in from the main flow path without irradiating the water to be treated with ultraviolet rays, and compares the amount of dissolved oxygen after irradiation and the amount of non-irradiated dissolved oxygen in the water to be treated flowing through the same position of the main flow path at the same timing, and measures an amount of at least one of an oxidizing agent and a reducing agent in the water to be treated flowing through the main flow path based on a comparison result.

### CROSS-REFERENCE TO RELATED APPLICATION

This application claims the benefit of priority of Japanese Patent Application No. 2023-081881 filed on May 17, 2023, the contents of which are incorporated by reference as if fully set forth herein in their entirety.

### BACKGROUND

### Technical Field

The present disclosure relates to a measurement device, a water treatment device, a measurement method, and a water treatment method.

### Related Art

Japanese Patent Application Laid-Open (JP-A) No. H03-278882 describes, as a method of removing dissolved oxygen in water, a configuration in which a reducing agent is dissolved in water in which oxygen is dissolved and ultraviolet rays are irradiated. However, it is not described that the reducing agent is quantified using the mechanism described in JP-A No. H03-278882.

In general, an oxidizing agent such as hypochlorous acid is added to raw water such as city water for the purpose of sterilization or the like. However, an amount of the oxidizing agent such as hypochlorous acid in raw water varies depending on the water quality of river water or the like and the operation status of a water purification plant. Therefore, in a case in which a reducing agent is added in order to remove the oxidizing agent in the water to be treated, it is necessary to change an addition amount of the reducing agent according to the amount of the oxidizing agent in the water to be treated, and it is important to easily grasp the amount of the oxidizing agent in the water to be treated. Furthermore, since an effective concentration of the reducing agent also decreases and a necessary amount of the reducing agent also changes with time, it is important to grasp the amount of the reducing agent after addition together.

However, in the conventional method in which the amount of the oxidizing agent is measured using a reagent, it takes time and effort to perform maintenance since the reagent is used, and a remaining amount of the oxidizing agent can be measured, but a remaining amount of the reducing agent cannot be measured. In a case in which the addition amount of the reducing agent is temporarily insufficient, for example, a reverse osmosis membrane in a subsequent stage may be deteriorated. Even if the subsequent stage is an ion exchange resin or the like, the same problem may occur. Furthermore, in a case in which an excessive amount of the reducing agent is added in order to prevent such a problem, a load of salt of equipment at the subsequent stage is caused, and not only there is a possibility that water quality is deteriorated, but also there is a possibility that running cost is increased.

### SUMMARY

An object of the disclosure is to provide a measurement device, a water treatment device using the measurement device, a measurement method, and a water treatment method capable of easily grasping an oxidation/reduction state of water to be treated.

A measurement device according to a first aspect includes: an ultraviolet irradiation unit capable of taking in part of water to be treated from a main flow path and irradiating the water to be treated with ultraviolet rays; a main dissolved oxygen measurement unit that measures an amount of dissolved oxygen after irradiation in the water to be treated, after irradiation with ultraviolet rays from the ultraviolet irradiation unit; a sub-dissolved oxygen measurement unit that takes in part of the water to be treated from the main flow path and measures an amount of non-irradiated dissolved oxygen in the water to be treated; a dissolved oxygen amount comparison unit that compares the amount of dissolved oxygen after irradiation with the amount of non-irradiated dissolved oxygen; a timing adjustment unit that adjusts the amount of dissolved oxygen after irradiation and the amount of non-irradiated dissolved oxygen to be compared in the dissolved oxygen amount comparison unit so as to be in the water to be treated flowing through a same position of the main flow path at a same timing; and an output unit that outputs a comparison result from the dissolved oxygen amount comparison unit.

In the measurement device of the first aspect, the main dissolved oxygen measurement unit measures the amount of dissolved oxygen after irradiation in the water to be treated after irradiation with ultraviolet rays by the ultraviolet irradiation unit, and the sub-dissolved oxygen measurement unit measures the amount of non-irradiated dissolved oxygen in the water to be treated that is not irradiated with ultraviolet rays. Then, the timing adjustment unit adjusts the amount of dissolved oxygen after irradiation and the amount of non-irradiated dissolved oxygen to be compared in the dissolved oxygen amount comparison unit so as to be in the water to be treated flowing through the same position of the main flow path at the same timing.

In a case in which an oxidizing agent is mixed in the water to be treated flowing through the main flow path, the dissolved oxygen is increased by the ultraviolet irradiation, and the amount of dissolved oxygen after irradiation is larger than the amount of non-irradiated dissolved oxygen in the comparison result output from the output unit. On the other hand, in a case in which the reducing agent is mixed in the water to be treated flowing through the main flow path, the dissolved oxygen is decreased by the ultraviolet irradiation, and the amount of dissolved oxygen after irradiation is smaller than the amount of non-irradiated dissolved oxygen in the comparison result output from the output unit. In a case in which neither the oxidizing agent nor the reducing agent is mixed in the water to be treated flowing through the main flow path, the amounts of dissolved oxygen of both are the same in the comparison result output from the output unit. Note that, in a case in which the oxidizing agent is present, dissolved oxygen increases, but an increase amount slightly varies depending on the type of the oxidizing agent, so that the quantitativity of the oxidizing agent is slightly inaccurate. Furthermore, in a case in which a reducing agent is present, dissolved oxygen decreases, but a decrease amount slightly varies depending on the type of the reducing agent, so that the quantitativity of the reducing agent is slightly inaccurate. However, under a condition where neither the oxidizing agent nor the reducing agent is mixed, that is, at an equivalent point, the dissolved oxygen ideally does not increase or decrease, but at this time, since the type of the oxidizing agent or the reducing agent is irrelevant, this condition can be accurately measured.

In this manner, it is possible to easily measure the oxidation state and the reduction state of the water to be treated based on the comparison result of the dissolved oxygen output from the output unit. In particular, it is possible to accurately measure the condition that the amounts of the oxidizing agent and the reducing agent are exactly equal and neither the oxidizing agent nor the reducing agent is mixed.

In the measurement device of the second aspect, the timing adjustment unit includes an adjustment chamber that equalizes a time until the water to be treated reaches the main dissolved oxygen measurement unit from a branch portion that branches from the main flow path, with a time until the water to be treated reaches the sub-dissolved oxygen measurement unit from the branch portion.

According to the measurement device of the second aspect, the adjustment chamber equalizes the time taken for the water to be treated to reach the main dissolved oxygen measurement unit from the branch portion and the time taken for the water to be treated to reach the sub-dissolved oxygen measurement unit from the branch portion, so that a simple configuration can be achieved.

Note that the measurement device may further include a sub-ultraviolet irradiation unit that is provided on an upstream side of the sub-dissolved oxygen measurement unit and is capable of irradiating the water to be treated with ultraviolet rays.

According to this configuration, the sub-ultraviolet irradiation unit irradiates the ultraviolet ray in the same manner as the ultraviolet irradiation unit, and measurement values of the main dissolved oxygen measurement unit and the sub-dissolved oxygen measurement unit at this time are matched, whereby zero matching (calibration) of both measurement units can be easily performed. Furthermore, problems such as a decrease in output of the ultraviolet irradiation of the ultraviolet irradiation unit and the sub-ultraviolet irradiation unit can be easily confirmed.

A water treatment device according to a third aspect includes: a measurement device according to the first aspect or the second aspect; a reducing agent addition unit that is provided on an upstream side with respect to a branch portion that branches from the main flow path and adds a reducing agent to the water to be treated; and a reducing agent amount adjustment unit that adjusts an amount of the reducing agent to be added by the reducing agent addition unit based on the comparison result output from the output unit.

According to the water treatment device of the third aspect, since the amount of the reducing agent to be added by the reducing agent addition unit is adjusted based on the comparison result output from the output unit, the amount of the reducing agent can be adjusted so that an amount of dissolved oxygen in the water to be treated flowing through the main flow path becomes a desired amount of dissolved oxygen.

A water treatment device according to a fourth aspect includes: an oxidizing agent addition unit that is provided on the upstream side with respect to the branch portion that branches from the main flow path and adds an oxidizing agent to the water to be treated; and an oxidizing agent amount adjustment unit that adjusts an amount of the oxidizing agent to be added by the oxidizing agent addition unit based on the comparison result output from the output unit.

According to the water treatment device of the fourth aspect, since the amount of the oxidizing agent to be added is adjusted by the oxidizing agent amount adjustment unit based on the comparison result output from the output unit, in a case in which the reducing agent remains in the water to be treated flowing through the main flow path, the reducing agent can be removed by adding the oxidizing agent.

A measurement method according to a fifth aspect includes: irradiating water to be treated taken in from a main flow path with ultraviolet rays, and then measuring an amount of dissolved oxygen after irradiation in the water to be treated; measuring an amount of non-irradiated dissolved oxygen in the water to be treated taken in from the main flow path without irradiating the water to be treated with ultraviolet rays; and comparing the amount of dissolved oxygen after irradiation and the amount of non-irradiated dissolved oxygen in the water to be treated flowing through a same position of the main flow path at a same timing, and measuring an amount of at least one of an oxidizing agent or a reducing agent in the water to be treated flowing through the main flow path based on a comparison result.

In the measurement method of the fifth aspect, the amount of dissolved oxygen after irradiation and the amount of non-irradiated dissolved oxygen in the water to be treated flowing through the same position of the main flow path at the same timing are compared. In a case in which an oxidizing agent is mixed in the water to be treated flowing through the main flow path, the amount of dissolved oxygen is increased by the ultraviolet irradiation, and the amount of dissolved oxygen after irradiation is larger than the amount of non-irradiated dissolved oxygen in the comparison result. On the other hand, in a case in which the reducing agent is mixed in the water to be treated flowing through the main flow path, the amount of dissolved oxygen is decreased by the ultraviolet irradiation, and the amount of dissolved oxygen after irradiation is smaller than the amount of non-irradiated dissolved oxygen in the comparison result. In a case in which neither the oxidizing agent nor the reducing agent is mixed in the water to be treated flowing through the main flow path, the amounts of dissolved oxygen of the oxidizing agent and the reducing agent are the same in the comparison result.

As described above, the oxidation state and the reduction state in the water to be treated can be easily measured based on the comparison result of the dissolved oxygen.

A water treatment method according to a sixth aspect includes: irradiating water to be treated taken in from a main flow path with ultraviolet rays, and then measuring an amount of dissolved oxygen after irradiation in the water to be treated; measuring an amount of non-irradiated dissolved oxygen in the water to be treated taken in from the main flow path without irradiating the water to be treated with ultraviolet rays; and comparing the amount of dissolved oxygen after irradiation and the amount of non-irradiated dissolved oxygen in the water to be treated flowing through the same position of the main flow path at the same timing, and adjusting an amount of at least one of an oxidizing agent or a reducing agent to be added to the water to be treated of the main flow path upstream with respect to a branch portion from the main flow path based on a comparison result.

In the water treatment method of the sixth aspect, the addition amount of at least one of the oxidizing agent and the reducing agent to the water to be treated in the main flow path is adjusted based on the comparison result between the amount of dissolved oxygen after irradiation and the amount of non-irradiated dissolved oxygen. As a result, the oxidizing agent or the reducing agent in the water to be treated flowing through the main flow path can be adjusted so as to have a desired amount.

In the technique of the disclosure, it is possible to, with respect to the water to be treated, easily grasp the oxidation state and reduction state of the water to be treated.

### BRIEF DESCRIPTION OF THE DRAWINGS

Exemplary embodiments of the present disclosure will be described in detail based on the following figures, wherein:

FIG. 1 is a configuration diagram illustrating an ultrapure water production system including a water treatment device of the present embodiment.

FIG. 2 is a configuration diagram illustrating a water treatment device of the present embodiment.

FIG. 3 is a configuration diagram illustrating a part of a measurement device of the present embodiment.

FIG. 4 is a configuration diagram illustrating a control system of a water treatment device of the present embodiment.

FIG. 5 is a flowchart of a reducing agent addition amount adjustment process.

FIG. 6 is a configuration diagram illustrating a part of a measurement device according to a modification example of the present embodiment.

FIG. 7 is a configuration diagram illustrating a water treatment device according to a modification example of the present embodiment.

FIG. 8 is an explanatory diagram in a case in which the measurement device of the present embodiment is used for other purposes.

### DETAILED DESCRIPTION

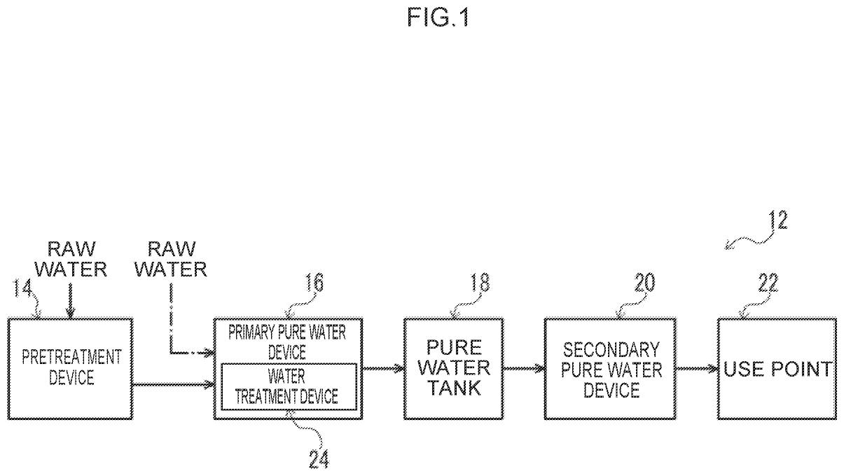

Hereinafter, a water treatment device 24 and an ultrapure water production system 12 according to a first embodiment will be described with reference to the drawings. As illustrated in FIG. 1, the ultrapure water production system 12 includes a pretreatment device 14, a primary pure water device 16, a pure water tank 18, a secondary pure water device 20, and a use point 22. The ultrapure water production system 12 is a system that removes impurities and the like from raw water to produce ultrapure water. Examples of the raw water include industrial water, tap water, groundwater, river water, and the like.

Raw water is supplied to the pretreatment device 14. In the pretreatment device 14, a treatment such as removal of turbidity is performed using activated carbon, a flocculation precipitation device, and a chromaticity removal device to obtain pretreatment water from which a part of suspended substances and organic substances in raw water are removed. Note that depending on the water quality of the raw water, the pretreatment device 14 may be omitted and the raw water may be sent to the primary pure water device 16 as indicated by a one-dot chain line in FIG. 1. As the pretreatment device, a sand filtering device, a pressurizing and floating device, or the like may be provided. The water to be treated (alternatively, raw water as water to be treated) treated by the pretreatment device 14 is sent to the primary pure water device 16. In a case in which an oxidizing agent such as hypochlorous acid in the raw water disappears or decreases in the stage of the treatment in the pretreatment device, the oxidizing agent can be added, if appropriate.

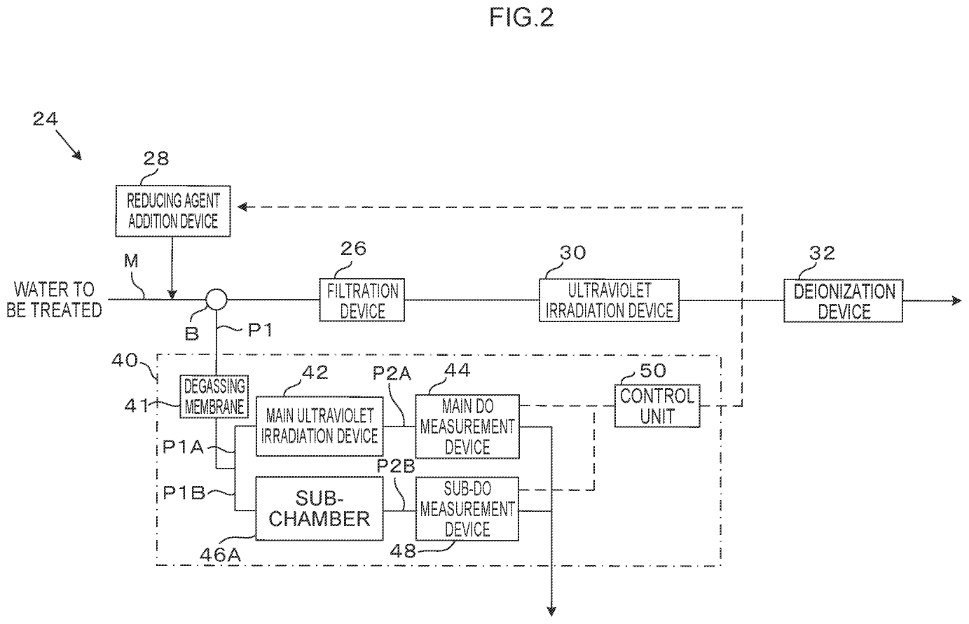

The primary pure water device 16 includes a water treatment device 24. As illustrated in FIG. 2, the water treatment device 24 includes a reducing agent addition device 28, a filtration device 26, an ultraviolet irradiation device 30, a deionization device 32, and a measurement device 40. The water to be treated is sent to the water treatment device 24 via a main flow path M.

The reducing agent addition device 28 can add a reducing agent to the water to be treated. In the present embodiment, sodium sulfite (Na₂SO₃), sodium bisulfite (NaHSO₃), sodium sulfamate (NH₂OSO₂Na), or the like is used as the reducing agent.

The filtration device 26 includes a reverse osmosis membrane therein. The filtration device 26 filters the water to be treated, whereby a part of foreign substances in the water to be treated is removed.

On a downstream side of the reducing agent addition device 28 and on an upstream side with respect to the filtration device 26, a branch portion B that branches part of the water to be treated from the main flow path M is provided. Part of the water to be treated is sent from the branch portion B to the measurement device 40 via a pipe P1. Details of the measurement device 40 will be described later.

The ultraviolet irradiation device 30 accelerates oxidative decomposition by irradiating the water to be treated to which the reducing agent is added with ultraviolet rays. The wavelength of the ultraviolet ray may be any wavelength as long as it can oxidize the water to be treated. For example, an ultraviolet irradiation device that emits ultraviolet rays having a wavelength of about 185 nm, which are generally used for ultraviolet oxidation, is preferable.

The deionization device 32 is provided downstream of the ultraviolet irradiation device 30, and removes ions and the like remaining from the water to be treated by ion exchange. However, depending on the type and state of the water to be treated, the deionization treatment may not be performed. Examples of the deionization device include, but are not limited to, an electrically regeneration-type ion exchange device, a mixed-bed ion exchange device (MB tower), and a boron-selective ion exchange resin tower. It is also possible to install a plurality of these in series. It is also possible to mix a boron-selective ion exchange resin in the mixed-bed ion exchange device (MB tower).

The primary pure water device 16 is a device that obtains primary pure water by removing impurities by performing a necessary treatment (cleaning treatment) on the water to be treated in this manner. That is, the primary pure water device 16 includes the water treatment device 24, and forms a pure water production device that obtains pure water from the water to be treated that has been treated by the water treatment device 24.

As the primary pure water device 16, for example, the following devices can be installed as necessary. Activated carbon tower, degassing tower, degassing membrane device, vacuum degassing tower.

The primary pure water obtained by the primary pure water device 16 is supplied to the pure water tank 18. The pure water tank 18 is a container that temporarily stores the primary pure water obtained by the primary pure water device 16.

The primary pure water stored in the pure water tank 18 is sent to the secondary pure water device 20.

The secondary pure water device 20 includes, for example, an ultraviolet irradiation device, a membrane degassing device, an ion exchange device (all not illustrated), and the like. The secondary pure water device 20 is a device that further performs a necessary treatment (cleaning treatment) on the water to be treated to remove impurities and obtains secondary pure water, that is, ultrapure water.

The obtained ultrapure water is sent to the use point 22 and used as cleaning water in, for example, a semiconductor manufacturing device.

### Measurement Device

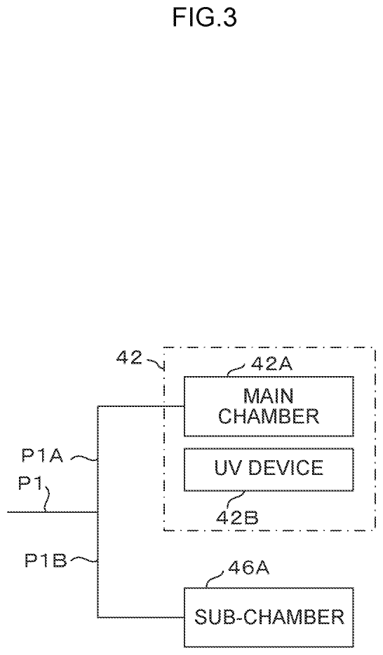

The measurement device 40 includes a degassing membrane 41, a main ultraviolet irradiation device 42, a main dissolved oxygen measurement device 44, a sub-chamber 46A, a sub-dissolved oxygen measurement device 48, and a control unit 50. As illustrated in FIG. 3, the main ultraviolet irradiation device 42 includes a main chamber 42A and a UV device 42B.

The water to be treated branched into the pipe PI at the branch portion B is sent to the degassing membrane 41. In the degassing membrane 41, dissolved oxygen in the water to be treated is adjusted, and the adjusted water to be treated is sent downstream. In the degassing membrane 41, nitrogen is injected in a case in which it is desired to lower the dissolved oxygen concentration. Furthermore, in a case in which the dissolved oxygen concentration is low, oxygen or air is injected. In any case, the amount of dissolved oxygen can be adjusted to a predetermined amount by adjusting an injection amount of nitrogen (or oxygen). The concentration of dissolved oxygen (Hereinafter, it may also be referred to as DO.) is preferably about 2 to 5 times with respect to the value of ΔDO to be controlled. Note that, in place of the degassing membrane 41, nitrogen (or oxygen) may be bubbled into the simple tank in order to adjust dissolved oxygen. Furthermore, in a case in which the variation of DO is large, the nitrogen (or oxygen) injection amount may be adjusted by an automatic valve so that the amount of nitrogen (or oxygen) can be automatically adjusted by the value of DO.

The water to be treated is further branched into two pipes P1A and P1B, and is delivered to the main chamber 42A and the sub-chamber 46A. A flow path from the branch portion B to the main chamber 42A and a flow path from the branch portion B to the sub-chamber 46A have the same diameter and the same length.

The main chamber 42A and the sub-chamber 46A can store the same volume of water to be treated, and are configured such that the water to be treated branched from the branch portion B at the same timing flows into the main chamber 42A and the sub-chamber 46A, respectively, and is sent out after the same time.

The UV device 42B can irradiate the water to be treated in the main chamber 42A with ultraviolet rays. Normally, the UV device 42B is in an on state (ultraviolet irradiation state). The UV device 42B is preferably a UV device that generates ultraviolet rays having a wavelength of about 254 nm used for sterilization, that is, a UV lamp for sterilization. Use of a UV device that generates ultraviolet rays having a wavelength of about 184 nm used for ultraviolet oxidation is not preferable because dissolved oxygen is reduced even without a reducing agent. Furthermore, the UV device may be a lamp filled with mercury or an LED lamp, and is not particularly limited.

The main chamber 42A and the main dissolved oxygen measurement device 44 are connected via a pipe P2A, and the sub-chamber 46A and the sub-dissolved oxygen measurement device 48 are connected via a pipe P2B. The pipe P2A and the pipe P2B have the same diameter and the same length, and are configured such that the water to be treated delivered from the main chamber 42A and the sub-chamber 46A at the same timing flows into the main dissolved oxygen measurement device 44 and the sub-dissolved oxygen measurement device 48 at the same timing.

That is, the water to be treated branched into the pipe P1 at the branch portion B is branched into two pipes P1A and P1B, one of which flows into the main dissolved oxygen measurement device 44 through the main chamber 42A and the pipe P2A, and the other flows into the sub-dissolved oxygen measurement device 48 through the sub-chamber 46A and the pipe P2B. The time until the water to be treated reaches the main dissolved oxygen measurement device 44 and the sub-dissolved oxygen measurement device 48 from the branch portion B is set to be the same time, and the water to be treated flowing through the main dissolved oxygen measurement device 44 and the sub-dissolved oxygen measurement device 48 at the same timing becomes the water to be treated flowing through the main flow path at the same timing.

The main dissolved oxygen measurement device 44 measures the dissolved oxygen in the flowed water to be treated and sends a measurement result to the control unit 50. Furthermore, the sub-dissolved oxygen measurement device 48 measures the dissolved oxygen in the flowed water to be treated and sends a measurement result to the control unit 50. Normally, the water to be treated to be measured by the main dissolved oxygen measurement device 44 has been irradiated with ultraviolet rays, and the water to be treated to be measured by the sub-dissolved oxygen measurement device 48 has not been irradiated with ultraviolet rays. The water to be treated after the measurement of dissolved oxygen is discharged to the outside from the main dissolved oxygen measurement device 44 and the sub-dissolved oxygen measurement device 48.

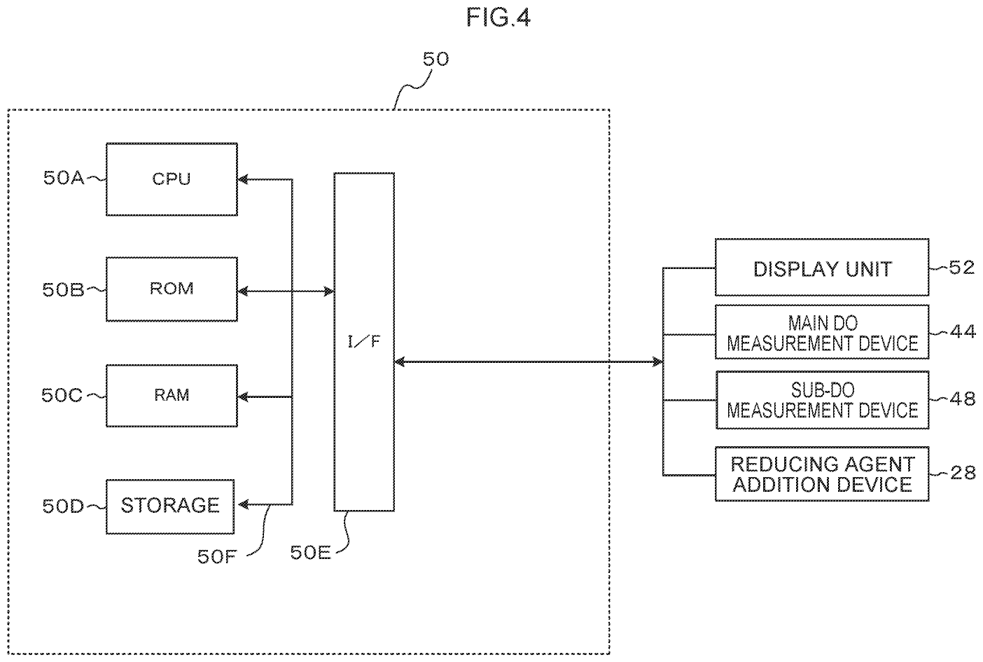

As illustrated in FIG. 4, the control unit 50 includes a CPU 50A, a ROM 50B, a RAM 50C, a storage 50D, an I/O 50E, and a bus 50F such as a data bus or a control bus that connects these components. The storage 50D stores a reducing agent addition amount adjustment program for adjusting the amount of the reducing agent to be added by the reducing agent addition device 28, various programs for controlling the ultrapure water production system 12, and data.

The main dissolved oxygen measurement device 44, the sub-dissolved oxygen measurement device 48, the reducing agent addition device 28, and the display unit 52 are connected to the I/O 50E. The control unit 50 adjusts the amount of the reducing agent to be added by the reducing agent addition device 28 based on the measurement results input from the main dissolved oxygen measurement device 44 and the sub-dissolved oxygen measurement device 48.

Next, a function and a water treatment method of the water treatment device 24 of the present embodiment will be described.

In the water treatment method using the water treatment device 24, the water to be treated pretreated by the pretreatment device 14 is sent to the water treatment device 24 of the primary pure water device 16 via the main flow path M. The reducing agent addition device 28 adds a preset initial amount of the reducing agent to the water to be treated flowing through the main flow path. The initial amount of the reducing agent is set so that a residual amount of the oxidizing agent after the addition of the reducing agent is reduced from the state of the conventional water to be treated, and the reducing agent does not remain in the water to be treated flowing into the filtration device 26.

The water to be treated to which the reducing agent has been added by the reducing agent addition device 28 is partially branched into the pipe Pl as sample water at the branch portion B and sent to the measurement device 40. The water to be treated of the sample water is branched into two pipes P1A and P1B, and is sent to the main chamber 42A and the sub-chamber 46A.

The water to be treated sent to the main chamber 42A is irradiated with ultraviolet rays emitted from the UV device 42B, sent to the main dissolved oxygen measurement device 44 through the pipe P2A, and the amount of dissolved oxygen is measured by the main dissolved oxygen measurement device 44. The water to be treated sent to the sub-chamber 46A is sent to the sub-dissolved oxygen measurement device 48 through the pipe P2B without being subjected to the ultraviolet irradiation treatment, and the amount of dissolved oxygen is measured by the sub-dissolved oxygen measurement device 48.

The amount of dissolved oxygen measured by the main dissolved oxygen measurement device 44 (hereinafter referred to as “value DO1 after ultraviolet irradiation”) and the amount of dissolved oxygen measured by the sub-dissolved oxygen measurement device 48 (hereinafter referred to as “ultraviolet non-irradiation value DO2”) are sent to the control unit 50.

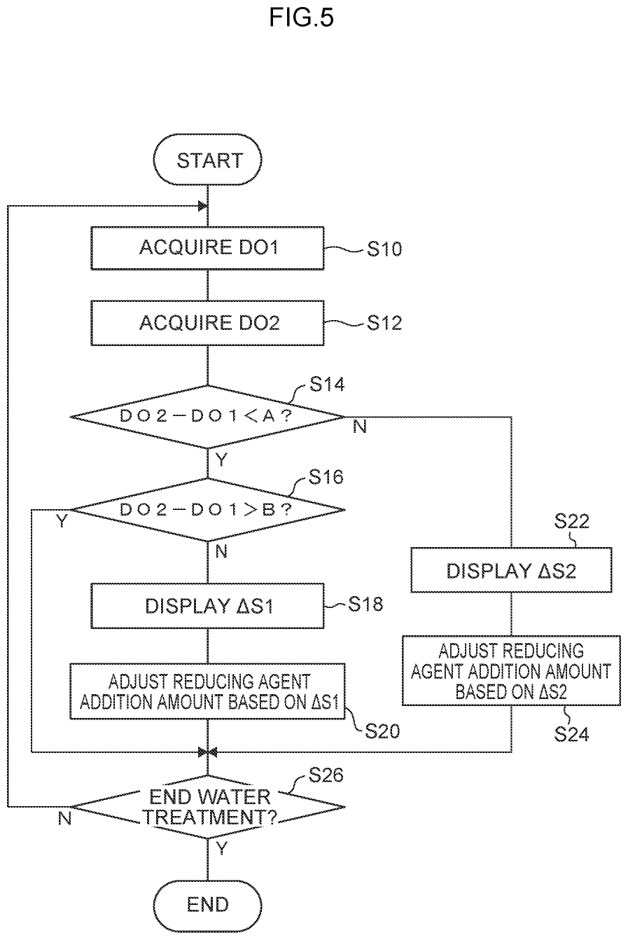

The control unit 50 executes a reducing agent addition amount adjustment process during operation of the water treatment device 24. As illustrated in FIG. 5, in the reducing agent addition amount adjusting process, the transmitted value DO1 after ultraviolet irradiation is acquired in step S10, and the transmitted ultraviolet non-irradiation value DO2 is acquired in step S12. In step S14, it is determined whether a difference (DO2−DO1) between the ultraviolet non-irradiation value DO2 and the value DO1 after ultraviolet irradiation is smaller than A set in advance. A is a higher numerical value for adjusting the difference between DO1 and DO2 to be a value of A to B in the reducing agent atmosphere, and is set in advance. The values of A and B can be arbitrarily set. As an example, A can be set to about 75 ppb, and B can be set to about 35 ppb. Within this range, a remaining amount of the oxidizing agent in the water to be treated is 0, and an excessive amount of the reducing agent is also about 0.5 ppm at the maximum.

Note that, in consideration of the influence on the reverse osmosis membrane in the subsequent stage, it is preferable that the atmosphere is slightly reduced, and thus A is preferably a value of DO2.

Furthermore, it is also possible to determine a set value of a difference between A and B, and to control the adjustment of the addition amount of the reducing agent such that the difference between A and B falls within a certain range. At this time, the set value of the difference between A and B can be set using a result of an experiment performed in advance, or the like.

In a case in which the determination in step S14 is negative, the value DO1 after ultraviolet irradiation is smaller than the ultraviolet non-irradiation value DO2, and there is a high possibility that the amount of dissolved oxygen in the water to be treated is decreased by the ultraviolet irradiation. This is because the oxidative decomposition of the water to be treated containing the reducing agent is promoted. Therefore, it can be seen that a predetermined or more reducing agent remains in the water to be treated which flows through the main flow path after the reducing agent is added by the reducing agent addition device 28 (excessive reducing agent). Accordingly, the process proceeds to step S22, a difference ΔS2 between DO2 and DO1 is output to the display unit 52 to be displayed, and in step S24, a signal is output to the reducing agent addition device 28 so that the addition amount of the reducing agent decreases according to ΔS2.

In a case in which the determination in step S14 is affirmative, it is determined in step S16 whether the difference between the ultraviolet non-irradiation value DO2 and the value DO1 after ultraviolet irradiation is larger than B set in advance. B is a lower numerical value for adjusting the difference between DO1 and DO2 (DO2−DO1) to be a value of A to B in the reducing agent atmosphere.

In a case in which the determination in step S16 is affirmative, the amount of dissolved oxygen in the water to be treated is within the range of A to B. In this case, since it is not necessary to adjust the addition amount of the reducing agent, the process proceeds to step S26.

In a case in which the determination in step S16 is negative, the value DO1 after ultraviolet irradiation is likely to be larger than the ultraviolet non-irradiation value DO2, and the amount of dissolved oxygen in the water to be treated is increased by the ultraviolet irradiation. Therefore, there is a high possibility that the oxidizing agent remains in the water to be treated which flows through the main flow path after the reducing agent is added by the reducing agent addition device 28 (oxidizing agent atmosphere). Accordingly, the process proceeds to step S18, a difference ΔS1 between DO2 and DO1 is output to the display unit 52 to be displayed, and in step S20, a signal is output to the reducing agent addition device 28 so that the addition amount of the reducing agent increases according to ΔS1.

After the addition amount of the reducing agent is controlled in steps S20 and S24, the process proceeds to step S26. In step S26, it is determined whether to end the water treatment in the water treatment device. In a case in which the water treatment is not ended, the process returns to step S10 and the above treatment is repeated. In a case in which the water treatment in water treatment device 24 is ended, the present treatment is ended.

In the measurement device 40 of the present embodiment, the state of the dissolved oxygen in the water to be treated is measured in this manner, the oxidation/reduction state can be displayed on the display unit 52, and the user can grasp the oxidation/reduction state of the water to be treated. Since the measurement here does not use a reagent, it does not take time and effort for maintenance as in the case of using a reagent.

Furthermore, by using the sub-chamber 46A, the time of outflow of the water to be treated irradiated with ultraviolet rays and the water to be treated not irradiated with ultraviolet rays is adjusted, so that the value DO1 after ultraviolet irradiation and the ultraviolet non-irradiation value DO2 can be compared for the water to be treated flowing through the main flow path M at the same timing. Note that, in the present embodiment, time adjustment is performed by using the sub-chamber 46A, but a time difference that occurs in a case in which the sub-chamber 46A is not used may be obtained in advance, and the value DO1 after ultraviolet irradiation and the ultraviolet non-irradiation value DO2 may be compared in consideration of this time difference.

Furthermore, since the measurement result is fed back to the reducing agent addition device 28 to adjust the addition amount of the reducing agent, the amount of the oxidizing agent in the water to be treated sent to the filtration device 26 can be reduced, and oxidation deterioration of the reverse osmosis membrane of the filtration device 26 can be suppressed. Moreover, the amount of the reducing agent in the water to be treated delivered to the filtration device 26 can also be reduced, and deterioration of the water quality and deactivation of a slime control agent in a downstream portion due to the residual reducing agent can be suppressed.

Note that, in the present embodiment, the dissolved oxygen of each of the water to be treated from the main chamber 42A and the water to be treated from the sub-chamber 46A is measured using two dissolved oxygen measurement devices of the main dissolved oxygen measurement device 44 and the sub-dissolved oxygen measurement device 48, but the dissolved oxygen may be measured using one dissolved oxygen measurement device. That is, the water to be treated from the main chamber 42A and the water to be treated from the sub-chamber 46A may be switched to each other by an automatic valve or the like to measure the value DO1 after ultraviolet irradiation and the ultraviolet non-irradiation value DO2.

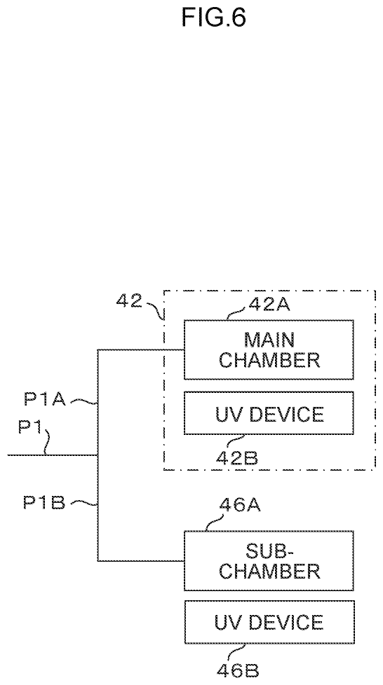

Note that, as a modification example of the present embodiment, as illustrated in FIG. 6, a UV device 46B that irradiates the sub-chamber 46A of the measurement device 40 with ultraviolet rays may be provided. The UV device 46B is not normally irradiated with ultraviolet rays, and can be used when the initial values of the main dissolved oxygen measurement device 44 and the sub-dissolved oxygen measurement device 48 are equalized. That is, by irradiating the main chamber 42A with ultraviolet rays from the UV device 42B and irradiating the sub-chamber 46A with ultraviolet rays from the UV device 46B in the same manner, and adjusting the main dissolved oxygen measurement device 44 and the sub-dissolved oxygen measurement device 48 so that the amount of dissolved oxygen measured by the main dissolved oxygen measurement device 44 and the amount of dissolved oxygen measured by the sub-dissolved oxygen measurement device 48 become the same, it is possible to perform zeroing, and it is possible to confirm a decrease in output and a defect of the ultraviolet device. Note that the zeroing can also be performed by turning off both the UV device 42B and the UV device 46B and adjusting the main dissolved oxygen measurement device 44 and the sub-dissolved oxygen measurement device 48.

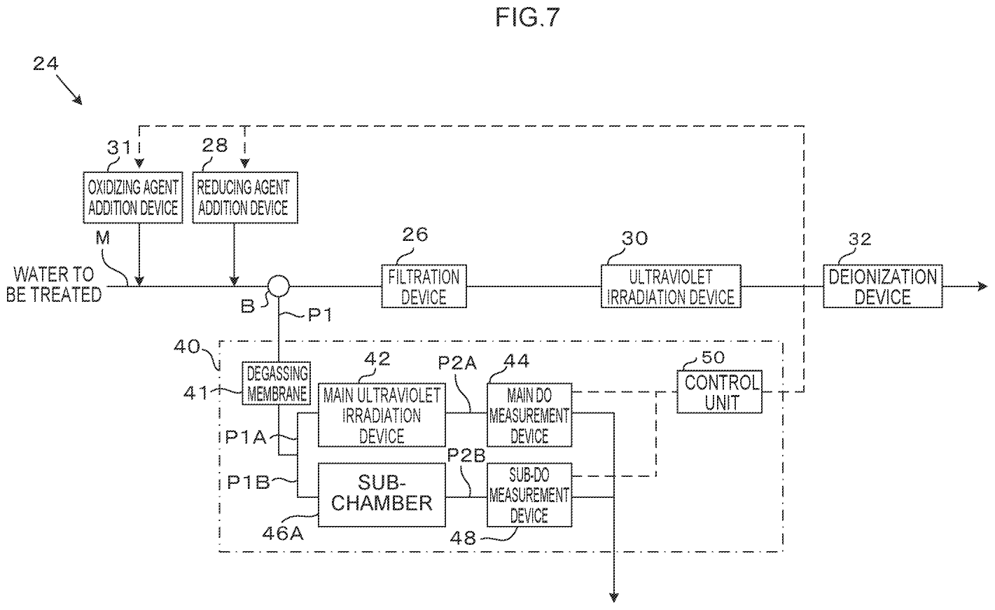

Furthermore, as illustrated in FIG. 7, an oxidizing agent addition device 31 may be provided on an upstream side of the main flow path M of the present embodiment. In this case, the oxidizing agent addition device 31 and the control unit 50 are connected, and the oxidizing agent can be added to the water to be treated according to the oxidation/reduction state in the measurement device 40.

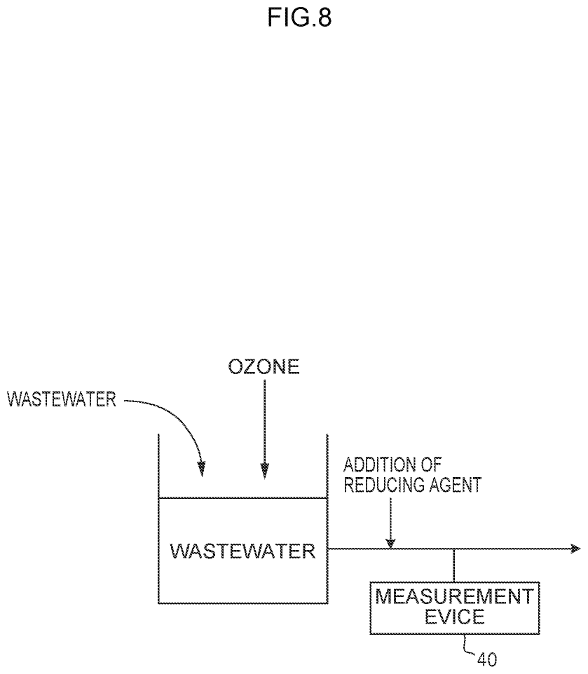

Furthermore, in the present embodiment, an example in which the measurement device 40 and the water treatment device 24 are applied to the ultrapure water production system 12 has been described, but the present invention can also be used for other purposes. For example, as illustrated in FIG. 8, when the wastewater of a semiconductor manufacturing factory or the like subjected to the oxidation treatment with ozone is subjected to the reduction treatment by adding a reducing agent, the oxidation/reduction state of the wastewater after the addition of the reducing agent can be monitored using the measurement device 40 to adjust the amount of the reducing agent to be added. In this case, the water quality (organic substance) amount of the wastewater increases or decreases according to the operation status of the semiconductor manufacturing factory. Therefore, since the amount of ozone remaining without being used for decomposition of the organic substance also increases or decreases, it is possible to optimize the amount of the reducing agent to be added and to prevent oxidation deterioration of an ion exchange resin device and the like installed at the subsequent stage.

Note that the present invention can be used not only for ozone oxidation but also for oxidation treatment using an oxidizing agent such as persulfuric acid, hypobromous acid, or hydrogen peroxide to adjust an amount of a reducing agent to be added at the subsequent stage.

### EXAMPLES

The measurement was performed under the following conditions by the measurement device 40 illustrated in FIG. 2.

Degassing membrane 41: SEPAREL PF-015 manufactured by DIC Corporation was used, and nitrogen gas was allowed to pass through at 0.05 NL/min.

Main ultraviolet irradiation device 30: Water was passed at a flow rate of 20 mL/min using a UV lamp SS801 (lamp power 8 W) manufactured by Sankyo Electric Co., Ltd. The irradiation amount is 3.6 kWh/m³. Furthermore, the same sub-chamber 46A and UV device 46B were used, and the UV device 46B was not turned on.

DO measurement devices 44 and 48: 510 type dissolved oxygen meter manufactured by HACK was used.

<Example 1> The RO permeated water produced by permeating city water through an RO membrane (low-pressure RO, 1st stage) was adjusted to DO200 ppb by the degassing membrane 41, and this was used as the water to be treated.

<Example 2 > Water obtained by adding 1 ppm of sodium sulfite to the water of Example 1 was used.

<Example 3 > Water obtained by adding 1 ppm of sodium hypochlorite to the water of Example 1 was used.

<Example 4> Water of Example 3 to which hypochlorous acid and an equivalent amount of sodium sulfite were added was used.

DO1: Measured value in main dissolved oxygen measurement device

DO2: Measured value in sub-dissolved oxygen measurement device

TABLE 1

| Examples | DO2 (ppb) | DO1 (ppb) | ΔDO (ppb) |
|---|---|---|---|
| 1 | 202 | 201 | −1 |
| 2 | 198 | 58 | −140 |
| 3 | 205 | 275 | +70 |
| 4 | 198 | 197 | −1 |

In Example 2, dissolved oxygen was reduced by 140 ppb with respect to 1 ppm of sodium sulfite. At this time, the amount of sodium sulfite is 1.1 ppm based on the following reaction Formula (A).

2SO₃²⁻+O₂→2SO₄²⁻ (A)

In Example 3, it is found that 70 ppb of dissolved oxygen was increased with respect to 1 ppm of sodium hypochlorite. Using a calibration curve obtained in advance (relationship between the concentration of sodium hypochlorite and an increase amount of dissolved oxygen), the concentration of sodium hypochlorite was 1.1 ppm.

In the case of Example 1 and Example 4, since the increase or decrease in the amount of dissolved oxygen is substantially zero, it can be seen that the amount of the oxidizing agent and the amount of the reducing agent are substantially equal even if the increase or decrease in the amount of dissolved oxygen is not converted into the amount of the oxidizing agent or the amount of the reducing agent by any method. Comparison between Example 1 and Example 4 shows that the amounts of hypochlorous acid and sulfurous acid are accurately measured to be equal.

What is claimed is:

1. A measurement device comprising:
an ultraviolet irradiation unit capable of taking in part of water to be treated from a main flow path and irradiating the water to be treated with ultraviolet rays;
a main dissolved oxygen measurement unit that measures an amount of dissolved oxygen after irradiation in the water to be treated, after irradiation with ultraviolet rays from the ultraviolet irradiation unit;
a sub-dissolved oxygen measurement unit that takes in part of the water to be treated from the main flow path and measures an amount of non-irradiated dissolved oxygen in the water to be treated;
a dissolved oxygen amount comparison unit that compares the amount of dissolved oxygen after irradiation with the amount of non-irradiated dissolved oxygen;
a timing adjustment unit that adjusts the amount of dissolved oxygen after irradiation and the amount of non-irradiated dissolved oxygen to be compared in the dissolved oxygen amount comparison unit so as to be in the water to be treated flowing through a same position of the main flow path at a same timing; and
an output unit that outputs a comparison result from the dissolved oxygen amount comparison unit.

2. The measurement device according to claim 1, wherein the timing adjustment unit includes an adjustment chamber that equalizes a time until the water to be treated reaches the main dissolved oxygen measurement unit from a branch portion that branches from the main flow path, with a time until the water to be treated reaches the sub-dissolved oxygen measurement unit from the branch portion.

3. A water treatment device comprising:
the measurement device according to claim 1;
a reducing agent addition unit that is provided on an upstream side with respect to a branch portion that branches from the main flow path and adds a reducing agent to the water to be treated; and
a reducing agent amount adjustment unit that adjusts an amount of the reducing agent to be added by the reducing agent addition unit based on the comparison result output from the output unit.

4. The water treatment device according to claim 3, further comprising:
an oxidizing agent addition unit that is provided on the upstream side with respect to the branch portion that branches from the main flow path and adds an oxidizing agent to the water to be treated; and
an oxidizing agent amount adjustment unit that adjusts an amount of the oxidizing agent to be added by the oxidizing agent addition unit based on the comparison result output from the output unit.

5. A measurement method comprising:
irradiating water to be treated taken in from a main flow path with ultraviolet rays, and then measuring an amount of dissolved oxygen after irradiation in the water to be treated;
measuring an amount of non-irradiated dissolved oxygen in the water to be treated taken in from the main flow path without irradiating the water to be treated with ultraviolet rays; and
comparing the amount of dissolved oxygen after irradiation and the amount of non-irradiated dissolved oxygen in the water to be treated flowing through a same position of the main flow path at a same timing, and measuring an amount of at least one of an oxidizing agent or a reducing agent in the water to be treated flowing through the main flow path based on a comparison result.

6. A water treatment method comprising:
irradiating water to be treated taken in from a main flow path with ultraviolet rays, and then measuring an amount of dissolved oxygen after irradiation in the water to be treated;
measuring an amount of non-irradiated dissolved oxygen in the water to be treated taken in from the main flow path without irradiating the water to be treated with ultraviolet rays; and
comparing the amount of dissolved oxygen after irradiation and the amount of non-irradiated dissolved oxygen in the water to be treated flowing through a same position of the main flow path at a same timing, and adjusting an amount of at least one of an oxidizing agent or a reducing agent to be added to the water to be treated of the main flow path upstream with respect to a branch portion from the main flow path based on a comparison result.
